(19) United States
(12) Patent Application Publication
(10) Pub. No.: US 2007/0160025 A1
(43) Pub. Date: Jul. 12, 2007

(54) Method for allocating reverse traffic channels at the time of handoff in a cdma communication system

(75) Inventors: Jung Hoon Lee, Seoul (KR); Hyun Gu Heo, Gyeonggi-do (KR)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: UTStarcom Korea Limited

(21) Appl. No.: 10/561,068

(22) PCT Filed: Jul. 26, 2004
(86) PCT No.: PCT/KR04/01882

§ 371 Date: Oct. 4, 2006

(30) Foreign Application Priority Data
Jul. 25, 2003 (KR) 10-2003-0051471

(51) Int. Cl. C07K 1/00; H04B 7/216; H04Q 7/00

(52) U.S. Cl. 370/342; 370/331

(57) ABSTRACT

A method for allocating reverse traffic channels at the time of handoff in a CDMA communication system, comprises the steps of: upon receiving a handoff request from a control station at a target base station, setting a Search Center Offset (SCO) to a Round Trip Delay (RTD) value received from a serving base station, and setting a searching area (S±β) to an area wider than a reference searching area (S) in view of an error (β) in the RTD value; dividing the searching area into a number of sub-areas, and allocating reverse traffic channels such that each reverse traffic channel covers its corresponding sub-area; monitoring the reverse traffic channels to determine whether a mobile station has been acquired; and if the mobile station has been acquired, releasing the remaining reverse traffic channels except for the reverse traffic channel which has acquired the mobile station.

### TECHNICAL FIELD

The present invention generally relates to a method for allocating reverse traffic channels at the time of handoff, and particularly to a method for allocating reverse traffic channels at the time of handoff in a Code Division Multiple Access (CDMA) communication system. More particularly, the method of the present invention increases the probability of a target base station acquiring a mobile station at the time of soft/hard handoff when the mobile station moves to the target base station from a serving base station by allocating a number of reverse traffic channels having their own search center offset and search window size.

### BACKGROUND ART

At the time of handoff in a conventional CDMA communication system, a target base station attempts to acquire a mobile station by allocating only one reverse traffic channel (shown in FIG. 1). The allocated reverse traffic channel has a search center offset and a search window size. The search center offset is a Round Trip Delay (RTD) value which is received from a serving base station that has been communicating with the mobile station.

However, since the RTD value is susceptible to the attenuation coefficient of the surrounding air, the RTD value cannot be always assumed to be exact. Thus, when the target base station allocates the reverse traffic channel by using the incorrect RTD value, signals from the mobile station cannot be received in the search window size. Therefore, the target base station's attempt to acquire the mobile station fails quite frequently, thereby resulting in call drops and deterioration of service quality.

### DISCLOSURE OF INVENTION

### Technical Problem

The object of the present invention is to provide a method for allocating reverse traffic channels at the time of handoff in a CDMA communication system. This can increase the probability of a target base station acquiring a mobile station at the time of soft/hard handoff, thereby reducing call drops and improving service quality.

### Technical Solution

To accomplish the above-mentioned object, there is provided a method for allocating reverse traffic channels at the time of handoff in a CDMA communication system comprising a control station, a serving base station and a target base station having a modem and a channel card. The method comprises the following steps of:

upon receiving a handoff request from the control station at the target base station, setting a Search Center Offset (SCO) to a Round Trip Delay (RTD) value received from the serving base station, and setting a searching area (S±β) to an area wider than a reference searching area (S) in view of an error (β) in the RTD value (step S1);

dividing the searching area (S±β) into a number of sub-areas, and allocating reverse traffic channels such that each of the reverse traffic channels covers its corresponding sub-area (step S2);

monitoring the reverse traffic channels to determine whether a mobile station has been acquired by any reverse traffic channel (step S3); and

if the mobile station has been acquired at the step S3, releasing the remaining reverse traffic channels except for the reverse traffic channel which has acquired the mobile station (step S4).

### BRIEF DESCRIPTION OF THE DRAWINGS

The above object and features of the present invention will become more apparent from the following description of the preferred embodiments given in conjunction with the accompanying drawings.

FIG. 1 shows a conventional method for allocating a reverse traffic channel at the time of handoff in a CDMA communication system.

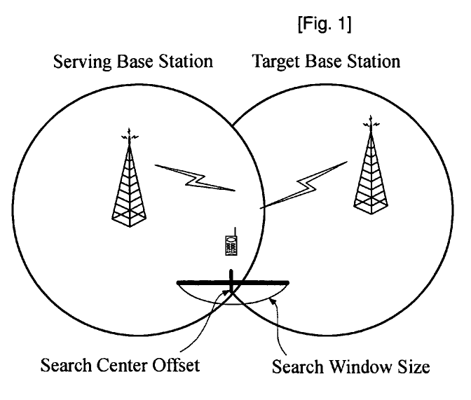

FIG. 2 is a block diagram showing a configuration of a device for locating reverse traffic channels at the time of handoff in a CDMA communication system in accordance with a preferred embodiment of the present invention.

FIG. 3 is a flowchart showing a method for allocating reverse traffic channels at the time of handoff in a CDMA communication system in accordance with a preferred embodiment of the present invention.

FIG. 4 shows the method for allocating reverse traffic channels at the time of handoff in a CDMA communication system in accordance with a preferred embodiment of the present invention.

### BEST MODE FOR CARRYING OUT THE INVENTION

The embodiment of the present invention will be readily understood as generally described and illustrated in the Figures provided herein and the accompanying text according to the above-identified technical scope of the present invention.

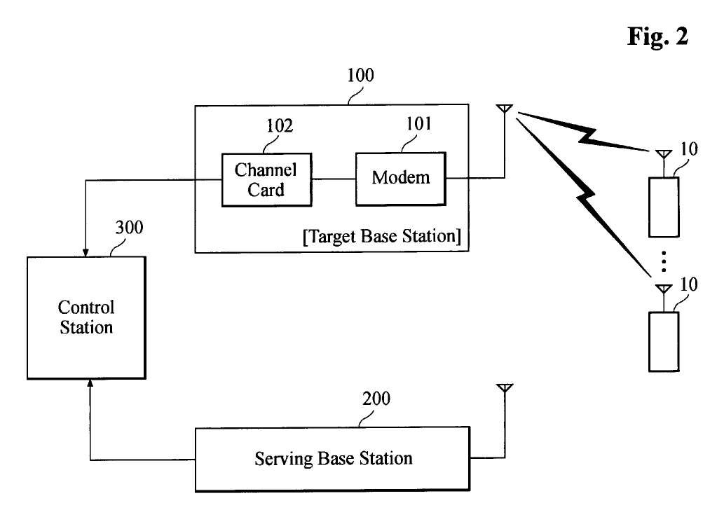

FIG. 2 is a block diagram showing a configuration of a device for allocating reverse traffic channels at the time of handoff in a CDMA communication system in accordance with a preferred embodiment of the present invention.

As shown in the drawing, a device for allocating reverse traffic channels at the time of handoff in a CDMA communication system according to the present invention comprises a target base station 100 having a modem 101 and a channel card 102, a serving base station 200, and a control station 300.

Upon receiving a handoff request from the control station 300, the modem 101 of the target base station 100 sets a Search Center Offset (SCO) to a Round Trip Delay (RTD) value received from the serving base station 200. It then sets a searching area (S±β) to an area wider than a reference searching area (S) in view of an error (β) in the RTD value.

Thereafter, the modem 101 (1) divides the searching area (S±β) into a number of sub-areas, (2) allocates a number of reverse traffic channels such that each of the reverse traffic channels covers its corresponding sub-area, and (3) monitors the reverse traffic channels to determine whether a mobile station 10 has been acquired by any reverse traffic channel. If the mobile station 10 has been acquired, then the modem 101 releases the remaining reverse traffic channels except for the reverse traffic channel which has acquired the mobile station 10.

Since the serving base station 200 is well known to those skilled in the art of CDMA communication system, the explanation thereof will be omitted.

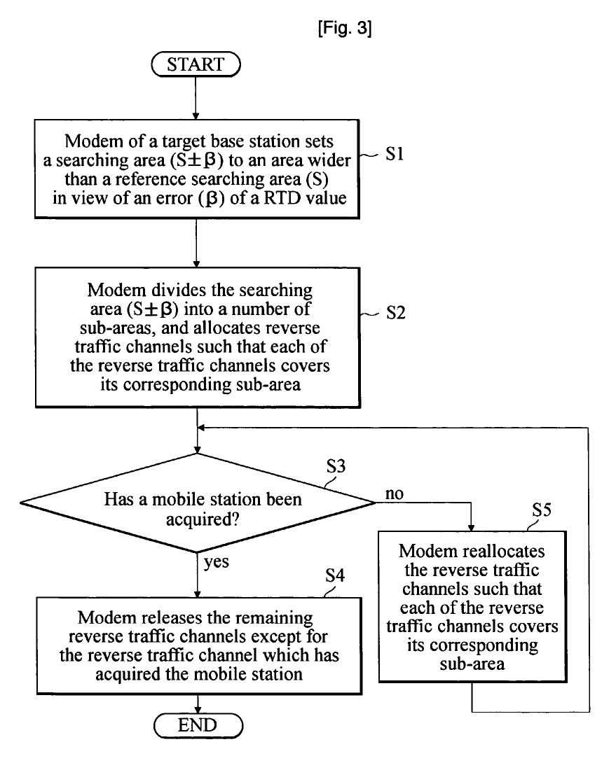

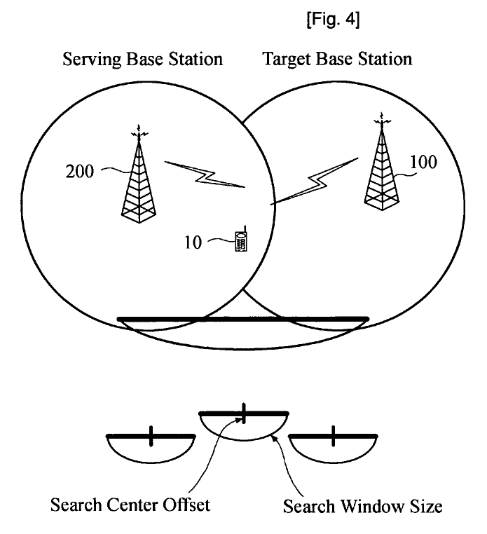

Hereinafter, a method for allocating reverse traffic channels at the time of handoff in a CDMA communication system according to the present invention will be described with reference to FIGS. 3 and 4.

Upon receiving a handoff request from the control station 300, the modem 101 of the target base station 100 sets the SCO to the RTD value received from the serving base station 200. Further, the modem 101 sets the searching area (S±β) to an area wider than the reference searching area (S) in view of the error (β) in the RTD value (step S1).

Then, the modem 101 divides the searching area (S±β) into a number of sub-areas and allocates a number of reverse traffic channels such that each of the reverse traffic channels covers its corresponding sub-area (step S2).

When allocating the reverse traffic channels at the step S2, the modem 101 sets the search center offsets and search window sizes, which are parameters of the reverse traffic channels, by the respective sub-areas. As shown in FIG. 4, this is so that the search window sizes partly overlap each other in order to cover the searching area thoroughly. The search window size of each reverse traffic channel is set to have a size sufficient to receive the multi-path of a base station cell coverage.

Then, the modem 101 monitors the reverse traffic channels to determine whether the mobile station 10 has been acquired by any reverse traffic channel (step S3).

If the mobile station 10 has been acquired at the step S3, then the modem 101 releases the remaining reverse traffic channels except for the reverse traffic channel which has acquired the mobile station 10 (step S4).

On the other hand, if the mobile station 10 has not been acquired at the step S3, then the modem 101 reallocates the reverse traffic channels such that each of the reverse traffic channels covers its corresponding sub-area. It then returns to the step S3 (step S5).

While the present invention has been shown and described with respect to a particular embodiment of a method for allocating reverse traffic channels at the time of handoff in a CDMA communication system, it will be apparent to those skilled in the art that many changes and modifications may be made without departing from the scope of the invention as defined in the appended claims and those equivalent thereto.

### INDUSTRIAL APPLICABILITY

As described above in detail a method for allocating reverse traffic channels at the time of handoff in a CDMA communication system according to the present invention can increase the probability of a target base station acquiring a mobile station at the time of soft/hard handoff when the mobile station moves to the target base station from a serving base station. This is achieved by allocating a number of reverse traffic channels having their own search center offset and search window size, thereby reducing call drops and improving service quality.

1. A method for allocating reverse traffic channels at the time of handoff in a CDMA communication system comprising a control station, a serving base station and a target base station having a modem and a channel card, said method comprising the steps of:
upon receiving a handoff request from the control station at the target base station, setting a Search Center Offset (SCO) to a Round Trip Delay (RTD) valve received from the serving base station, and setting a searching area (S±β) to an area wider than a reference searching area (S) in view of an error (β) in the RTD value (step S1);
dividing the searching area (S±β) into a number of sub-areas, and allocating reverse traffic channels such that each of the reverse traffic channels covers its corresponding sub-area (step S2);
monitoring the reverse traffic channels to determine whether a mobile station has been acquired by any reverse traffic channel (step S3); and
if the mobile station has been acquired at the step S3, releasing the remaining reverse traffic channels except for the reverse traffic channel which has acquired the mobile station (step S4).

2. The method of claim 1, wherein if the mobile station has not been acquired at the step S3, the method further comprises the step of reallocating reverse traffic channels such that each of the reverse traffic channels covers its corresponding sub-area and returning to the step 3 (step S5).

3. The method of claim 1, wherein when allocating the reverse traffic channels at the step S2, the search center offsets and search window sizes, which are parameters of the reverse traffic channels, are set by the respective sub-areas such that the search window sizes partly overlap each other so as to cover the searching area thoroughly.

4. The method of claim 3, wherein the search window size of the each reverse traffic channel is set to have a size sufficient to receive a multi-path of a base station cell coverage.
